(19) United States
(12) Patent Application Publication
(10) Pub. No.: US 2010/0291536 A1
(43) Pub. Date: Nov. 18, 2010

(54) SAMPLE PROCESSING CASSETTE, SYSTEM, AND METHOD

(75) Inventors: Hendrik J. Viljoen, Lincoln, NE (US); Scott E. Whitney, Lincoln, NE (US); Joel R. Termaat, Lincoln, NE (US); Alison Freifeld, Omaha, NE (US); Elsje Pienaar, Lincoln, NE (US)

Correspondence Address: DOBRUSIN & THENNISCH PC, 29 W LAWRENCE ST, SUITE 210, PONTIAC, MI, 48342, US

(73) Assignee: Streck, Inc., LaVista, NE (US)

(21) Appl. No.: 12/780,345

(22) Filed: May 14, 2010

Related U.S. Application Data
(60) Provisional application No. 61/216,360, filed on May 15, 2009.
(60) Provisional application No. 61/216,225, filed on May 14, 2009.

(51) Int. Cl. C12P 19/34; C12N 1/06; C12Q 1/00; C12M 1/00

(52) U.S. Cl. 435/4; 435/259; 435/91.2; 435/283.1; 435/289.1

(57) ABSTRACT

The present invention provides a method and device for collecting treating and analysis of biological or chemical material by introducing a source material into a specimen container, transferring the source material to a processing device and thermally, chemically and/or mechanically treating the source material to alter at least one constitutive characteristic of the source material and to release or create a target material from the source material.

### CLAIM OF PRIORITY

This application claims the benefit of the filing date of U.S. Provisional Application Ser. Nos. 61/216,360, filed on May 15, 2009 and 61/216,225, filed on May 14, 2009 the entirety of the contents of these applications being hereby incorporated by reference for all purposes.

### FIELD OF THE INVENTION

This invention relates to integrated processing units for the collection, processing and analysis of a source material.

### BACKGROUND OF THE INVENTION

The collection and testing of source material, including biological or chemical specimens, presents a number of challenges, especially in locations without sufficient health, forensic and laboratory resources. Accurate disease diagnosis typically requires laboratory facilities that include advanced testing equipment and skilled laboratory technicians. Unfortunately, many areas that experience high rates of infectious disease mortality do not have the funding, infrastructure, or skilled labor force necessary to establish or maintain such facilities. As a result, disease diagnosis in these areas is often unreliable or unavailable, leading to ineffective treatment and the inability to contain diseases. Even in highly developed communities, the fragile nature of many types of source materials require that the materials are either tested for disease presence immediately or preserved until arrival at an adequate laboratory facility. Thus, when a source material cannot be immediately tested, there is a considerable amount of time between source material collection and eventual diagnosis, where an individual may be unknowingly transmitting a disease to others.

As a result, without the convenience of a nearby laboratory facility, disease diagnosis is generally facilitated by obtaining a source material (e.g., a biological specimen which may include blood, saliva, sputum, tissue, feces, urine, semen, vaginal secretions, hair, tears, cerebral fluid, spinal fluid, bone material or the like) from a patient at a remote site and then sending the source material to a centralized laboratory for testing. During transfer of source material samples, the sample may degrade or be damaged to the point where accurate diagnosis is improbable or even impossible upon arrival at a laboratory facility. Even if a source material is received in an acceptable condition, days or even months may pass before a patient receives a diagnosis. In some areas, it may be challenging to locate and notify a patient of a positive diagnosis, only adding to the difficulty of controlling the spread of communicable diseases in these areas.

Disease control has been of particular concern for areas with high rates of tuberculosis. The spread of tuberculosis faces a number of obstacles given the ease with which it is transmitted and the vast number of individuals who are carriers of the disease but are asymptomatic. Tuberculosis is generally an airborne bacteria that is easily spread through close contact. As an additional obstacle, many regions having a high prevalence of tuberculosis also have high rates of HIV/AIDS. Immunocompromised patients have an increased likelihood of developing active and/or drug-resistant tuberculosis, and are more difficult to diagnose, which in turn leads to a substantially higher rate of mortality. Thus, accurate diagnosis and treatment in these areas with large HIV/AIDS populations is critical.

As an added difficulty, the standard tests for tuberculosis diagnosis in many areas includes smear microscopy and mycobacterial culture. While sensitive, culture typically requires six weeks or more to obtain growth and identification of the mycobacteria and/or drug susceptibility. While relatively inexpensive, smear microscopy is reported to identify only half the cases of tuberculosis (even less for HIV/AIDS co-infection) and is also unable to identify if a strain is drug-resistant. Thus, the current systems for tuberculosis diagnosis leads to low rates of disease identification in a timely and accurate manner, thereby limiting patient follow-up and proper treatment. These consequences perpetuate not only spread of the disease, but also the development of drug-resistant strains of tuberculosis.

Existing polymerase chain reaction (PCR) technology has also been used for the diagnosis of tuberculosis, but has been hindered by its highly complex preparative steps and long amplification times in the range of hours. In many clinical settings, typical diagnostic methods (including PCR) are comprised of a considerable number of steps and a considerable number of lab devices to prepare and analyze the sample to obtain an actual diagnostic result. While there have been advances in the sample collection to results process (typically by consolidating and automating certain steps), the fact remains that molecular diagnostics are typically confined to high-complexity labs. Even where PCR testing has been shown somewhat effective, most health care facilities cannot support the funding or staffing needs for an operational PCR lab. Additionally, the expense and complexity of conventional PCR technology has prohibited it from being widely applied for diagnosis in areas where tuberculosis is most prevalent. The cost requirements for a high complexity laboratory simply cannot be met in many remote, underdeveloped or economically struggling areas.

In response, there has been a push for point-of-care diagnostic devices that will accurately diagnose tuberculosis while substantially reducing the time required for diagnosis. However, point-of-care diagnostics for tuberculosis pose additional challenges. The risk of infection for any health care worker or lab technician is extremely high with tuberculosis samples. Most laboratories that regularly handle infected tuberculosis samples are equipped with fume hoods, biohazard safety cabinets, air sanitation systems or isolated rooms so that anyone in contact with the samples is at reduced risk for infection. Health facilities that would generally be expected to serve as point-of-care testing locations are often simply not equipped to handle these types of infectious source materials. Further, current PCR diagnostics require expensive machinery and/or have slow processing times which make existing PCR technologies unsuitable for point-of-care use in some areas. Thus, any point-of-care device should also minimize the need for high-technology equipment and technicians.

Notwithstanding the above, there remains a need for point-of-care diagnostic equipment that reduces the risk of infection to health-care workers, improves the accuracy and speed of diagnostic results, and does so with simplified low-cost equipment. There is a further need for diagnostic equipment that provides an accurate diagnosis while a patient is still located at the point-of-care facility so that infected individuals can be treated immediately to help reduce the risk of infecting others. This accurate diagnosis should also provide data regarding drug-resistant strains of a disease so that patients are not treated with a medication to which they are resistant. Proper medication will reduce the risk of transmission to others and reduce the spread of drug resistance from overuse/misuse of antibiotic drugs. There is also a need for diagnostic equipment that provides a closed system where health care workers have preferably no direct contact with any source material. There is a further need for diagnostic equipment having low-cost, simplified components so that the equipment can be easily repaired in developing areas.

The present invention addresses the above needs by providing a point-of-care diagnostic device that provides quick and accurate disease diagnosis that includes a closed system and low-cost, simplified components. The present invention further provides for collection, treatment and analysis of a source material wherein the source material is collected and sealed and transferred to a processing device where it is thermally, chemically and/or mechanically treated, and transferred to an analysis location. The present invention further draws upon kinetics to not only automate steps that are typically performed manually, but to reduce the overall processing time. The present invention provides that all collection, treatment and analysis steps may take place at one point-of-care facility so that patients can receive accurate diagnosis information quickly allowing treatment to begin immediately, thus reducing the risk of transmitting the disease to others.

### SUMMARY OF THE INVENTION

The present invention provides a method for preparing biological or chemical material by introducing a source material into a specimen container, transferring the source material to a processing device and thermally, chemically and/or mechanically treating the source material to alter at least one constitutive characteristic of the source material and to release or create a target material from the source material. As referred to herein, constitutive characteristics of a source material may include one or more characteristics of the source material, and may include a physical characteristic, a chemical characteristic, or both. It may include one or more of a composition, a concentration, a chemical reaction, a mechanical characteristic, a morphological characteristic, a rheological characteristic, an electrical characteristic, an optical characteristic, a magnetic characteristic, a thermal characteristic, or any combination thereof. Any altering of a constitutive characteristic may be irreversible, or alternatively, a source material may undergo additional treatment to reverse or modify the alteration of a constitutive characteristic. In the context of a particular source material (e.g., sputum), the material may be processed for altering one or more rheological characteristics.

The present invention further provides for a processing device for use with source materials comprising a mixing portion, a transport means, at least one interface for a control device and a covering means. A source material may be introduced into the mixing portion where it may be treated to alter at least one constitutive characteristic of the source material and to release or create a target material from the source material. The transport means may transfer the target material between the mixing portion and an amplification/detection portion. The at least one interface for a control device may control the temperature, mixing operation, transport, or any combination thereof. The covering means may enclose the mixing portion.

The present invention also contemplates a processing device for use with source materials comprising a body configured to include a processing well, a fluid transport path, at least one heating element, a temperature sensing device and a covering. The processing device may also include a cooling device. The processing well may be adapted to receive a device for mixing and pumping a source material. The fluid transport path may include a valve. The at least one heating element may be disposed proximate the processing well. The temperature sensing device may be disposed proximate the processing well. The covering may be placed over the processing well so that the contents of the processing well remain within the body.

The invention herein contemplates a device and method for the collection, treatment and analysis of a source material wherein all collection, treatment and analysis steps may take place at one point-of-care medical facility. The diagnostic equipment disclosed herein may allow for the collection, treatment and analysis of the source material to be performed in a closed system with minimal transfer of source material and minimal technician participation so that risk of infection to health care workers is minimized. Collection of source material may occur so that the source material is sealed within a specimen container. The collection may be followed by source material treatment, transfer, amplification and/or detection. The treatment may occur so that the source material releases or creates a target material for analysis. All collection, treatment, and analysis may occur in a shortened time frame so that patients can provide a sample and receive a diagnosis in one trip to a health care facility.

### BRIEF DESCRIPTION OF THE DRAWINGS

FIG. 1 is a perspective view of an illustrative processing device in accordance with the present invention.

FIG. 2A is a cross sectional view of the processing device shown in FIG. 1 having a motor within a mixing well.

FIG. 2B is a cross sectional view of a processing device in accordance with the present invention having a motor external to a mixing well.

FIG. 3 is top-down view of an additional embodiment of a processing device in accordance with the present invention.

FIG. 4 is top-down view of an additional embodiment of a processing device in accordance with the present invention.

FIG. 5 is side view of an additional embodiment of a processing device in accordance with the present invention.

### DETAILED DESCRIPTION

In general, the invention herein contemplates a device and method for the collection, treatment and analysis of a source material. In the application to clinical diagnostics, all collection, treatment and analysis steps may take place at one point-of-care medical facility. The processing equipment disclosed herein allows for simultaneous mechanical, chemical and/or thermal treatment of a source material. All collection, treatment and analysis of the source material may be performed in a closed system so that risk of exposure of workers to the source material or any derivative of the source material is minimized. The collection may occur so that the source material is sealed within a specimen container. The treatment may occur so that the source material releases or creates a target material for analysis. For example, a lysing step may be employed by which a cell well or cell membrane is degraded to release one or more nucleic acids and/or proteins contained therein. Complete processing, amplification and/or analysis may occur in a shortened time frame (e.g., less than about 5 hours, less than about 2 hours, less than about 1 hour, or even less than about 0.5 hours). For example, patients can provide a sample and receive a diagnosis in one trip to a health care facility. The present invention has particular applicability and is used for testing source materials for diagnosing a disease and/or drug-resistant strains of a disease, or any other health condition. As an example, the present invention may be used to detect multiple drug-resistant tuberculosis (MDR-TB), extensively drug-resistant tuberculosis (XDR-TB), drug-resistant Clostridim difficile, methicillin-resistant staph aureus (MRSA), vancomycin intermediate staph aureus (VISA), vancomycin-resistant staph aureus (VRSA), or the like. The present invention may further be useful in other applications of testing of source materials (such as forensic testing or bacterial/fungal testing), which may include or be obtained from textiles, soil, food, water, mold scrapings, swabs, or the like.

More particularly, the present invention provides a processing device (e.g., a processing cassette). The processing device may receive a source material (e.g., a biological or chemical specimen) in a processing well where the source material is treated mechanically, thermally and/or chemically. The source material may be subsequently transferred (to a location within or external to the processing device) and amplified. The source material may be treated mechanically, thermally and/or chemically in a simultaneous manner (e.g., within the processing device) to reduce the time-frame of treatment and to reduce handling of the source material by health care workers. The treatment of the source material may include chemical modification of the source material's rheology to promote flow and mixing, lysis of cells to release DNA, RNA, proteins and/or antigens, reduction of reaction (e.g., PCR) inhibitors from the source material and/or transfer of the source material or a portion of the source material to an amplification and/or detection location.

The source material may include blood, saliva, sputum, tissue, feces, urine, semen, vaginal secretions, hair, tears, biopsy material, cerebral fluid, spinal fluid, bone material or any other biological or chemical sample that may be tested for disease presence. In addition to health care-related specimens, the present invention is useful for testing other specimens or source materials (which may include or be found in textiles, soil, food, water, and mold). The mechanical processing may include a mixing member and motor for mixing a source material in the processing well (e.g., mixing well). The thermal processing may include active temperature control (e.g., active heating and/or active cooling via fan and/or pettier device) of the source material to one or more elevated and/or lowered temperatures. The chemical processing may include contacting the source material with one or more chemical agents. Each of the mechanical, thermal and/or chemical processing steps may modify the source material so that the source material or a portion of the source material is formatted for accurate analysis. The formatting process may include steps to reduce the viscosity of a source material, lyse the cells within a source material, protect the cells from unwanted nuclease and/or protease effects, or any other treatment so that any eventual analysis of the source material or a portion of the source material will be facilitated and/or improved (e.g., by resolving inconsistencies with the composition of the source material). The mechanical, chemical, and/or thermal treatment may cause a source material to release or create a target material that may be contained within the source material prior to treatment. Each of the mechanical, chemical and/or thermal treatment steps may assist in extracting a target material from a source material. The target material may include DNA, RNA, proteins, antigens, serum, cells, plasma, contaminants, reaction products, hybridization targets, water, pollutants or any combination thereof.

The present invention further contemplates a system wherein a plurality of wells are included within one processing device. Each well may include a mixing member as necessitated by the particular task. Thus, the mechanical, chemical and/or thermal processing and any transfer, amplification and/or detection steps may occur at the same stage or time frame in each successive well. For example, each well may be simultaneously thermally treating the samples within each of the plurality of wells. Alternatively, each well may be engaging in a different segment of the process at each well. As an example, one well may be chemically treating a source material and another well may be amplifying a sample. As another example, one well may be mechanically, chemically, and/or thermally treating a source material and another well may be used for subsequent real-time PCR.

As stated above, the mechanical processing may include the use of a mixing member located in a mixing well (e.g., processing well or mixing portion). The mixing member may include an impeller structure that facilitates both mixing of a source material in the well and pumping of the source material or a portion of the source material out of the well. The mixing member may cyclically impinge upon the source material to alter or facilitate alteration of at least one constitutive characteristic of the source material. The mixing member may be reciprocally activated, rotationally activated or a combination thereof through a number of cycles.

The mixing member may be interchangeable with other mixing members of differing shape depending upon the composition of the source material. As an example, the mixing member may include a relatively wide profile for a source material having a relatively high viscosity or a more thin profile for a source material having a lower viscosity. As an example, the mixing member may have a thickness of at least about 0.1 mm. The mixing member may have a thickness of less than about 5 mm, less than about 2 mm, or even less than about 0.5 mm. The length of the mixing member may depend upon the size and/or diameter of the mixing well in which the mixing member is located. The length of the mixing member may be less than the diameter of the mixing well so that the mixing member can move (e.g., spin and/or oscillate) within the well without contacting or with minimal contact with the inner wall of the mixing well. The length of the mixing member may be at least about 0.5 mm. The length of the mixing member may be less than about 20 mm, less than about 5 mm, or even less than about 1 mm.

The mixing member or any other portion of the processing device including any well, transport means, or amplification/detection portion, may have or be treated to have desired hydrophobicity or hydrophilicity characteristics. Within the mixing well, this may promote a source material to move toward or away from the mixing member thereby increasing turbulence within the mixing well. The mixing member may mix the source material with any chemical processing agents that are added to or pre-loaded within the processing device. The mixing function may promote increased contact between the source material and any chemical processing agents so that a desired effect (e.g., a chemical reaction, cell lysis, mucolysis, hybridization, diffusion) is enhanced within the source material. The mixing member may include a dual-functional head configured for homogenizing and/or reducing the viscosity of the source material (e.g., by application of shear forces) and for pumping some or all of the source material to a downstream location. For example, a mixer may include an impeller having an attached member that is cycled in one direction for altering a material characteristic (e.g., reducing viscosity) and also cycled in another direction where it applies a force (e.g., as a pump) for pushing or otherwise transferring the source material. The mixing well may further include one or more protrusions extending from a wall of the mixing well into the well to promote turbulence within the mixing well.

The mixing member may be integrated with or separable from the motor. For example, the motor may include a shaft to which an impeller is attached. The shaft may be separable from the impeller or integrally formed therewith. The mixing member, motor or both may be disposable and/or potentially autoclavable. The disposable nature of all or a portion of the mixing well, including the mixing member and motor, may assist in reducing cross-contamination and/or improving the overall safety of the diagnostic equipment in that no cleaning of the mixing well or its contents would be required which would further reduce the risk of source material contact for health care workers. Thus, methods herein contemplate that they may be free of any cleaning step during handling of a single source material, or even between the handling of two or more successive source materials.

The motor may be included to cause the mixing member to spin, oscillate, cycle or have any similar motion that imparts shear to a source material, causes turbulence within a source material, or both. The mixing member may be an impeller structure having a shaft portion that contacts or nearly contacts at least one wall of the mixing well. For example, a sleeve and/or bearing may be present between the wall and the shaft such that the shaft may rotate freely while providing a fluid tight seal. Alternatively, the shaft may contact or almost contact the mixing well through the covering means. For example, the shaft portion may be integrated into a hinged lid. The motor may include an output shaft that engages an input shaft of the mixing member or mixing well. The spinning of the mixing member and any attached shafts or structural members may promote increased mixing and contacting rates with any chemical agents located within the well. The spinning may also reduce the viscosity of the source material and may accelerate any hybridization reactions within the mixing well (such as DNA, RNA or protein hybridization to probes or affinity media). The motor may be activated by a controller which may control the torque and/or direction of the mixing member. The desired torque of the mixing member may be driven by the viscosity of the source material. Advantageously, a source material having a higher viscosity may be mixed at a higher torque to effectively break down the source material. A source material having a lower viscosity may be mixed at a lower torque as the break down process is minimized. The mixing member may be a magnetized impeller that is activated by magnetic field manipulation proximate the mixing well. The movement (e.g., spinning) of the mixing member may impart heat to a source material.

The mixing well may also include a cover. The cover may prevent the source material from exiting the well during the mixing process to provide additional protection to health care workers. The cover may cover only the mixing well, or may cover other portions of the processing device as well. The cover may only cover the mixing well, wherein other portions of the processing device are pre-sealed. The processing device may include a cover on the mixing well and an additional cover for the entire processing device. The cover may be threaded so that it may be securely screwed onto the mixing well. The cover may include a hinged or sliding lid. The cover may be fastened to the mixing well via a mechanical engagement (e.g., an interlock, a friction fit, or other interference fit), an adhesive attachment, or any combination thereof. The cover may be a flexible material that adheres to the mixing well via an adhesive or may be mechanically attached to the well. The cover may or may not be removable. The cover may have an internal flap for safe sample loading.

The mixing well may also include a pumping mechanism for pumping all or a portion of a source material out of the mixing well. The pumping mechanism may also include a structural member that may be the mixing member. The structural member may move in one direction (e.g., counter-clockwise) for mixing purposes and the opposite direction (e.g., clockwise) for pumping purposes. The pumping mechanism may also employ pressure gradients to assist the source material in moving into and/or out of the mixing well. The pumping mechanism may pump the source material or a portion of the source material into an amplification well. A detection well may be similarly used or the detection step integrated into the amplification well (e.g., by real-time PCR). The pumping mechanism may pump the source material or a portion of the source material through a transport path to the amplification well. The source material or a portion of the source material may be transported from the mixing well to another location by capillary forces (e.g., by wicking).

The pumping mechanism may pump the source material or a portion of the source material through a transport path. Amplification and/or detection may occur in the transport path, thus removing the need for an amplification well. In the event that amplification and/or detection occurs in the transport path, a waste well may collect any remaining source material after the source material has undergone amplification and/or detection in the transport path. It may also be possible that the processing device includes a well for DNA amplification that is downstream of the mixing well so that the source material is transported via the transport path from the mixing well to the well for DNA amplification. Further, a separate well for reverse transcription of RNA may be included within the processing device, or external of the processing device. The mixing well may also include one or more entry and/or exit ports for the entry and exit of source material, target material, chemical processing agents, or any combination thereof.

In addition to mechanical processing via mixing, the processing device may further process a source material by contacting the source material with one or more chemical processing agents. The chemical processing agents may be added to the source material to prepare the source material for amplification and/or detection and to cause the source material to release or create a desired target material. The chemical processing agents may be added to the mixing well prior to addition of the source material. The chemical processing agents may be added to the source material prior to, during or after any mixing step. The chemical processing agents may be added to the source material prior to, during or after any heating and or cooling step. Depending upon the composition of the source material, the chemical processing agents contacted with the source material may differ. It is possible that the chemical processing agents may be stored within the processing device. The chemical processing agents, which may be pre-sealed during manufacture, may be located in a reagent well or reservoir prior to contact with a source material. The processing device may thus include a channel that transfers the chemical processing agents from the reagent well to the mixing well for treatment of a source material. It is possible that the chemical processing agents may be pre-loaded within the mixing well prior to entry of the source material into the mixing well. The chemical processing agents may also be contacted with the source material or target material within the transport means. The chemical processing agents may include an additional mixer and/or pumping mechanism in the reagent well.

The chemical processing agents may include one or more of a variety of agents such that the selection of the appropriate agents will depend upon the composition of the source material and the desired function of the agent within the source material. A nuclease inhibitor may also be present to protect the DNA from damage from any nucleases that may be present in the source material. For example, in the event that a source material treatment protocol is performed so that a source material releases DNA as a target material, the chemical processing agents may include a lysis buffer to promote cell lysis so that cellular DNA (the target material in this case) is released as a result of the cell lysis process. The type of chemical processing agents that may be used include but are not limited to reducing agents, nuclease inhibitors, enzymes, lysis buffers, protease inhibitors, phosphatase inhibitors, metabolic inhibitors, enzyme inhibitors, fixatives (e.g., protective agents), acids, bases, organic solvents, alcohols, drying agents, water, heavy water, mucolytic agents, sterilizers or any combination thereof.

As an example, source material samples (in liquid or even solid form) having a higher viscosity (e.g., a viscosity of at least about 2 Pa·s, at least about 5 Pa·s, at least about 20 Pa·s, or even at least about 100 Pa·s) may require contacting with a chemical processing agent that will assist the mixing function (including chopping of solid source materials) in breaking down the source material to reduce the viscosity. A reducing agent may be used as a viscosity-reducing agent (e.g., a mucolytic agent). The reducing agent may be capable of breaking down the chemical structure of one or more molecules that make up the source material. Specifically, the reducing agent may be capable of reducing the disulfide bonds of proteins and to prevent further forming of disulfide bonds between protein residues. The thermal processing steps described herein may further allow the reducing agent to contact the source material under denaturing conditions caused by the application of high heat so that any disulfide bonds that may be inaccessible at room temperature may be effectively accessed and reduced. Examples of such reducing agents include but are not limited to dithiothreitol (DTT), mercaptoethanol, mercaptoethylamine, Tris[2-carboxyethyl]phosphine (TCEP), (N-acetylcysteine), Nacystelyn, dornase alfa, thymosin β₄, guaifenesin, or any combination thereof. The concentration of reducing agent for reducing the viscosity of the source material may be at least about 1 mM. The concentration of reducing agent may be less than about 50 mM. The concentration of reducing agent may be from about 5 mM to about 25 mM. It may be less than about 30 mM, less than about 20 mM, or even less than about 10 mM. One preferred approach is to use about 10 mM to about 20 mM DTT.

The concentration of reducing agent may be low enough that effective PCR amplification and analysis will not be inhibited. One unique aspect of the teachings is that the preferred use of certain viscosity-reducing agents surprisingly may be employed advantageously despite their known tendency to potentially compromise PCR amplification.

A nuclease inhibitor may also be added to the source material. The nuclease inhibitor desirably is one or more agents that are used in sufficient quantity so as to protect any target nucleic acids from deleterious nucleases. The nuclease inhibitor may also prevent inhibition of molecular beacons used for nucleic acid detection. The nuclease inhibitor may act to prevent DNase and/or RNase activity within the source material. The nuclease inhibitor is preferably present in an amount sufficient to prevent a decrease in the amount and/or quality of the target material recoverable from the source material as compared with a sample that does not include a nuclease inhibitor. Examples of such nuclease inhibitors include but are not limited to diethyl pyrocarbonate, ethanol, aurintricarboxylic acid (ATA), formamide, vanadyl-ribonucleoside complexes, macaloid, ethylenediamine tetraacetic acid (EDTA), proteinase K, heparin, hydroxylamine-oxygen-cupric ion, bentonite, ammonium sulfate, dithiothreitol (DTT), beta-mercaptoethanol, cysteine, dithioerythritol, tris(2-carboxyethyl)phosphene hydrochloride, a divalent cation such as Mg⁺², Mn⁺², Zn⁺², Fe⁺², Ca⁺², Cu⁺² and any combination thereof. For example, the amount of nuclease inhibitor added to the source material may be at least about 10 μg/ml. The amount of nuclease inhibitor added to the source material may be less than about 200 μg/ml. The amount of nuclease inhibitor added to the source material may be from about 50 μg/ml to about 150 μg/ml. The amount of nuclease inhibitor may be less than about 130 μg/ml, less than about 100 μl/ml, or even less than about 70 μg/ml.

Some nuclease inhibitors may also degrade proteins, including those necessary for amplifying the target material. It is possible that the methods herein may employ one or more steps of deactivating any nuclease inhibitor. For instance, the source material may require additional thermal and/or chemical treatment in order to deactivate the nuclease inhibitor. As an example, an additional step of heating the source material to a temperature of about 90° C. to about 105° C. after contact with the one or more chemical processing agents may be employed to deactivate Proteinase K prior to amplification.

In order to stimulate release or creation of a target material, it may also be desirable to lyse cells located within the source material. The methods herein thus may include one or more steps of stimulating release or creation of target material including one or more lysis steps. The lysing may include treating physically and/or thermally for rupturing a cell wall or membrane so that cell contents are expelled from within the cell. One approach contemplates chemically treating a source material with an agent such as a lysis buffer. A lysis buffer may thus be added to the source material. Examples of lysis buffers that may be used include but are not limited to tris-HCl, EDTA, tris-EDTA, EGTA, SDS, deoxycholate, TritonX, NaCl, sodium phosphate, NP-40, phosphate buffered saline (PBS) and combinations thereof. The concentration of lysis buffer for lysing cells within the source material may be at least about 0.25 mM or even 5 mM. The concentration of lysis buffer may be less than about 30 mM or even 20 mM. The concentration of lysis buffer may be from about 1 mM to about 20 mM. As an example, the source material may be contacted by a lysis buffer including from about 0.5 mM to about 5 mM EDTA and from about 5 mM to about 15 mM Tris-HCl. The lysis buffer may include from about 0.5 mM to about 5 mM EDTA and from about 5 mM to about 15 mM Tris-HCl at a concentration of at least about 1×. The lysis buffer may include from about 0.5 mM to about 5 mM EDTA and from about 5 mM to about 15 mM Tris-HCl at a concentration of less than about 100×. The lysis buffer may include from about 0.5 mM to about 5 mM EDTA and from about 5 mM to about 15 mM Tris-HCl at a concentration of about 15× to about 25×.

In the event that source materials are treated to test for tuberculosis, sufficient cell lysis may be employed so that the dense cell wall of the mycobacteria typically associated with tuberculosis is broken down. The cell lysis process may include multiple steps designed to break down the highly crosslinked peptidoglycan structure of the mycobacteria cell wall. The lysis process may be specialized to include critical levels of heat and critical amounts and types of chemical lysis buffers that have been identified to weaken the mycobacteria cell wall quickly to the point of rupture. As an example, a lysis buffer having a concentration of from about 5× to about 30×0.5 mM to about 10 mM EDTA and about 1 mM to about 20 mM Tris-HCl may be utilized to have an increased rate of mycobacteria cell wall weakening.

As discussed above, the effective processing of a source material may include one or more steps of thermal processing. Active temperature control of the mixing well may facilitate increased reaction and diffusion kinetics. The source material and any chemical processing agents may be added to the mixing well and the source material may be mixed by the mixing member. Since viscoelastic materials may have viscosity that depends upon the shear rate of the material, the mixing action of the mixing member may aid the processing by temporarily lowering the viscosity of the source material. Prior to mixing, during mixing or after mixing, the temperature of the mixing well may be raised and/or lowered for thermal treatment of the source material. The thermal treatment may aid in mucolysis by modifying a physical characteristic of the source material. The thermal treatment may also promote cell lysis. In the event that the source material being treated is sputum, a temperature increase may promote mucolysis by generally reducing the viscosity of the source material. However, if the sputum temperature becomes too high, the sputum proteins will denature and the sputum will become dehydrated resulting in an undesirable increase in viscosity. Thus, any thermal treatment of sputum must be precise and carefully monitored through a temperature sensor, at least one heater and/or at least one cooling device.

The mixing well may be heated to an initial starting temperature of at least about 30° C. The initial starting temperature may be less than about 60° C. The initial starting temperature may be from about 35° C. to about 45° C. The initial starting temperature may be about 37° C. The mixing well may then be quickly heated to a desired lysis and mucolysis temperature. The lysis and mucolysis temperatures may be the same in that both mucolysis and lysis may occur once the mixing well is raised to a predetermined temperature. Alternatively, the mixing well may be raised to a first temperature for mucolysis purposes and a second temperature for lysis purposes. The lysis and/or mucolysis temperature may be at least about 35° C. The lysis and/or mucolysis temperature may be less than about 110° C. The lysis and/or mucolysis temperature may be about 70° C. to about 95° C. The lysis and/or mucolysis temperature may be about 90° C. The lysis and/or mucolysis temperature may be less than any temperature where the heat begins to deleteriously affect the source material thereby reducing the accuracy of any detection and/or PCR results. The lysis and/or mucolysis temperature may vary depending upon the composition of the source material.

As an example, the lysis and/or mucolysis temperature may be higher for source materials having a higher relative viscosity (e.g., sputum). The lysis and/or mucolysis temperature for sputum may be at least about 80° C., and more preferably about 90° C. Similarly, the lysis temperature for source materials containing mycobacteria (which are often sputum samples) may also be increased to reflect the difficulty in rupturing the mycobacteria cell wall. The lysis processes described herein may include thermal and chemical treatment so as to reduce the mycobacteria cell wall thickness by about 10% to about 20%, preferably about 14% to about 17% in order to promote cell lysis. Such lysis temperatures may be at least about 80° C., and more preferably about 90° C. The chemical and thermal treatments disclosed herein may promote sufficient lysis for amplification and/or detection within about 50 to about 600 seconds at such temperatures. Sufficient lysis may occur in less than about 500 seconds, less than about 300 seconds, or even less than about 100 seconds. The source material may also be subjected to additional temperature increases to deactivate any chemical processing agents that may interfere with a later amplification and/or detection steps. The source material may be subjected to further thermal treatment during amplification. For example, any PCR processing may subject the source material or a portion of the source material to multiple temperature increases.

Thermal processing may take place by way of a holding device into which the mixing well may be placed that may provide both heat for thermal processing and the motor for the mixing structure. The holding device may include an opening for receiving the mixing well. The mixing well may be permanently attached to and/or integrally formed with the holding device. The mixing well may instead be removable from the holding device. As an example, a disposable mixing well may be removable from the holding device so that it is not necessary for the entire holding device to be disposable. Alternatively, the mixing well and holding device may both be disposable. The holding device may further include one or more conductive (e.g., highly thermally conductive) walls that contact the opening for receiving the mixing well. The one or more conductive walls may be composed of one or any combination of conductive materials including but not limited to silver, copper, aluminum, gold, brass, rhodium, platinum, titanium, highly thermally conductive polymer materials, or any combination thereof.

The holding device may also include a means for providing heat to the mixing well via the one or more conductive walls. The means for providing heat may be connected to a power source (e.g., a DC or AC power source) that provides electricity for heat production. The power source may be a battery located within the holding device or located external to the holding device. The power source may originate from an analysis and/or amplification device. The holding device may be powered by solar power. The means for providing heat may include thermoelectric devices, resistive heaters, power resistors, other types of heating devices or any combination thereof. The means for providing heat may also provide a cooling function to remove heat from the mixing well or any other portion of the processing device. Cooling may also be provided by a fan device.

The means for providing heat to the mixing well may include one or more temperature sensors for monitoring the temperature of the conductive walls, the mixing well, the source material, or any combination thereof. The one or more temperature sensors may be in direct contact and/or thermal communication with a source material. The one or more temperature sensors may include a resistance temperature device (RTD), thermistor, thermocouple, or infrared scanner. The one or more temperature sensors may be in direct contact with a wall that contacts a source material. It may also be possible that the one or more temperature sensors may employ non-contact temperature detection (e.g., IR thermography). The means for providing heat to the mixing well may include a temperature control for raising and lowering temperature of the conductive walls, the mixing well, the source material, or any combination thereof as required by any thermal treatment specifications. As an example, the temperature sensor may determine if the temperature of the mixing well and/or its contents should be raised or lowered to reach a starting temperature, an elevated temperature, a mucolytic temperature or a lysis temperature. A multitude of temperature set points and the times at each can be programmed. The temperature set points and times can be cycled through at least one heater and optional cooler to promote processes such as amplification of the biological target material. Alternatively, more than one chamber may be present for processing, each at its own isothermal set point and the fluid contents transferred among the chambers. The temperature sensor and temperature control may be integrated into one device that both controls and senses the temperature. The temperature sensor and temperature control may be separate devices. One or both of the temperature sensor and temperature control may be located within the holding device, or even within the mixing well. One or both of the temperature sensor and temperature control may be located external to the holding device but having a portion connected to the holding device for accurate temperature measurement and temperature control. The heaters and temperature sensors may take on a substantially cylindrical shape or any other shape that may minimize the space required for the heaters and sensors and/or maximize contact with one or more portions of the processing device.

The temperature control may require manual adjustment to the temperature or may be modified automatically according to a pre-programmed thermal treatment protocol. The thermal treatment protocol may be programmed via software that may be integrated within the holding device or may be part of a computing or control device located external from the holding device. The temperature control and/or thermal treatment protocol may be modified according to the composition of the source material. For example, a source material having a higher viscosity may require exposure to higher temperatures or exposure to greater number of variable temperatures in an effort to reduce the viscosity of the sample. As a specific example, the thermal processing of mycobacteria-containing samples may include processing at higher temperatures to rupture the dense cell wall of the mycobacteria.

The holding device may also include additional components. The processing device may include portions composed of conductive (e.g., highly thermally conductive) materials and portions composed of poor thermally conductive materials. The holding device may include an insulating material located beyond and in contact with the one or more conductive walls to maintain heat within the holding device. The insulating material may surround each of the one or more conductive walls or may surround only a portion of the one or more conductive walls. The poor thermally conductive materials may be in contact with a conductive material. The poor thermally conductive materials may provide insulation to the conductive material so that at least some of the heat provided to the processing device or a portion of the processing device will be maintained within the processing device. The poor thermally conductive materials in contact with the conductive material may also have a melting point that is sufficiently high so that the poor thermally conductive material does not degrade or melt when heat is applied to the conductive materials. The processing device may experience temperatures as high as at least 80° C., at least 100° C. or even as high as 115° C. so that any poor thermally conductive material that is in contact with the conductive material may not degrade at such temperatures. The insulating material may be a glass, porcelain, paper-based material, or any polymeric material including but not limited to thermoplastics, thermoset plastics, elastomeric containing materials or any combination thereof. Examples of polymeric and elastomeric materials that may be employed include PTFE, PEEK, delrin, nylon, polyvinyl chloride, polypropylene, high-density polyethylene, low-density polyethylene, linear low-density polyethylene, polyvinylidene chloride polyamide, polyester, polystyrene, polyethylene, polyethylene terephthlate, bio-based plastics/biopolymers (e.g., poly lactic acid), silicone, acrylonitrile butadiene styrene (ABS), rubber, polyisoprene, butyl rubber, polybutadiene, EPM rubber, EPDM rubber, or any combination thereof.

The processing device may also include a controller that is integrated with the processing device, separate from the processing device, or integrated with a separate amplification and/or detection device. The controller may be in communication with and may control thermal devices (e.g. resistive heaters, thermoelectric modules), motors and temperature sensors to operate the components of the processing device and perform a protocol input by a user via an interface. A central processing unit may be tasked with executing a predetermined protocol. One or more H-bridges may be useful for alternating the impeller direction or controlling the heating and/or cooling of any thermoelectric modules. Digital or analog outputs may be employed to turn on and/or turn off the motor, heaters, coolers and control the amount of voltage/current applied thereto. An analog-to-digital converter may be utilized in processing the signal from the temperature sensor. The controller may also include a display of the protocol status, including temperature, motor speed (e.g., torque), and progress may be displayed numerically and/or graphically by the display. It is possible that a bench-top instrument accompanies the processing device.

As previously mentioned, in an effort to reduce risk to health care workers, the holding device may also include a covering. The covering may cover only a portion of the holding device or may cover the entirety of the holding device. As will be discussed further herein, the holding device, the mixing well or both may include a port that allows for the collection of the source material within the mixing well. The port may also be sealed to avoid exposure to the source material during transfer into the mixing well.

The source material may be placed into the mixing well via any process that maintains the closed system attributes described herein. It is possible that, the source material is collected from a patient into a specimen container. Examples of suitable specimen containers are described in U.S. Provisional Application No. 61/216,225 filed May 14, 2009 and a commonly owned co-pending U.S. Application entitled SPECIMEN CONTAINER, SYSTEM, AND METHOD to Viljoen et al. being filed on May 14, 2010, the same day as the present application, both applications being incorporated by reference herein for all purposes. The specimen container may include a receiving portion and an opening. The specimen container may be adapted to be hermetically sealed after receiving the source material so that the source material is isolated within the container. Upon receiving a source material, the specimen container may be sealed to define one or more compartments within the specimen container so that the source material is only located in one portion (e.g., one compartment) of the specimen container. The one or more compartments may be separated from one another prior to or during transfer of the source material into the processing device.

The specimen container may include an expulsion portion in fluid communication with the receiving portion into which the source material is transferred within the specimen container. The specimen container may further include an expulsion port through which the source material is expelled from the container. The expulsion port may be adapted for being sealingly interfaced (e.g., connected) with the processing device and optionally for detachment from the expulsion portion following expulsion of the source material into the processing device. The mixing well (e.g., mixing portion) may include one or more interior wall surfaces adapted for being sealingly interfaced with the specimen container. The mixing well may be threaded so that it may be connected to the specimen container during transfer of the source material to the mixing well and may also connect to a threaded cap to cover the mixing well during source material treatment. At least a portion of the receiving portion may include a flexible wall structure that contacts the source material.

It is possible that, pressure may be applied for displacing at least a portion of the source material from the receiving portion to the expulsion portion. The pressure may be applied by a manual and/or automated rolling or pressing device or may be applied manually by a health care worker. The expulsion portion may also include a flexible wall structure that contacts the source material and to which pressure is applied for displacing at least a portion of the source material from the expulsion portion through the expulsion port and into the source material processing device.

Several safety features may be built into the sample processing device. The main safety feature includes the separation of the sample from the device users. The processing device may be pre-sealed or enclosed and a cover, if any, may shut or seal tightly to minimize the chance of leakage. The cover itself may have a dual enclosure feature similar to well designed inflatable (e.g. a beach ball or an air mattress) where an outer cover seals tightly and an inner flap is sealed only when the sample is supposed to go through a transport path. Entry ports from the specimen container to the processing device may be sealed with heat and/or pressure to make a tight seal and to destroy potential chemical/biological hazards in the sealed region. Automation of the processing steps may reduce the need for human interaction and potential human errors when handling the source material and biological target material. The transport means may avoid the common use of centrifuges, thereby eliminating the risk of exposure in the rare but typically violent failure of the centrifuge. An optional ultraviolet light (typically in the 200 nm to 300 nm wavelength range) can be incorporated into the processing device to aid in destruction of any potential hazardous materials. The inexpensive and disposable nature of the processing device and mixers may allow for economical and safe disposal such as incineration and/or autoclave treatment of the processing device. Optional temperature sensitive paint, temperature sensitive wax, and/or a temperature film gauge can be applied to the outside of the processing device for quick visual inspection to ensure that the processing device has reached the proper temperature(s) during processing. Failsafe components may be included such as heaters that turn off automatically in the case of an equipment failure. Combined, these safety features may allow for minimal exposure of the user to any potentially hazardous contamination.

The amount of source material that may be received from the specimen container into the processing device may be at least about 2 μl. The amount of source material that may be received from the specimen container into the processing device may be less than about 4000 μl. The amount of source material that may be received from the specimen container into the processing device may be from about 250 μl to about 2000 μl. The amount of source material amplified may be the same as the amount received into the processing device or may be substantially less than the amount of source material received into the processing device. As an example, the initial amount of source material received by the processing device may be about 500 μl. Upon cell lysis and release of the target material, an aliquot of the mixing well contents may be transferred to an amplification well via a transfer means in an amount of only about 3 μl to about 50 μl. The amount of target material amplified may be less than about 40 μl, less than about 30 μl, less than about 20 μl, or even less than about 10 μl.

After transfer of the source material from the specimen container to the mixing well, the source material may be treated mechanically, chemically and/or thermally as described herein. After treatment, the source material or a portion of the source material (e.g., the target material) may be processed, amplified, detected, or any combination thereof. The amplification may allow for the detection of the presence or absence of particular genetic or disease related sequences. The amplification process may occur in the mixing well. In order to amplify only the target material, it may be necessary to remove any remaining source material (e.g., waste material) from the mixing well. A wash step may be incorporated to further remove any remaining source material. This removal may be performed by pumping the waste material from the mixing well into an additional well or elsewhere. As previously discussed, the pumping mechanism may be facilitated by the mixing member. The mixing member may spin in the opposite direction of that used for mixing (e.g., the mechanical treatment) for pumping purposes. The target material may be transferred from a first portion of the processing device to a second portion of the processing device that is spaced apart from the first portion but in fluid communication with the first portion.

As discussed herein, the amplification process may take place in a second location (e.g., the amplification portion). The amplification portion may be located within the processing device or may be located external from the processing device. The second location may be an internal amplification well, tube, path or channel located within the processing device. The second location may be an external amplification well located external from the processing device.

The processing device may also include a transport means for transferring at least a portion of the source material to the amplification portion. The transport means may also facilitate the transfer of one or more substances throughout (e.g., within, into or out of) the processing device. The transport means may include a fluid transport path or tube. The transport means may include a capillary portion. The transport means may include a valve for controlling the transport function so that fluid flow may be stopped, slowed or otherwise controlled. The valve may be opened and/or closed automatically or manually.

The transport means include one or more channels or valves through which fluid and/or air returns back to the mixing portion. Thus the transport means, the mixing portion, or both may further include a pressure release portion to facilitate effective transport of source material within the processing device. The processing device or a component of the processing device may include a means for introducing a pressure gradient so that a first portion of the processing device has a first pressure and a downstream portion of the processing device has a second pressure that is lower than the first pressure. As an example, the mixing well may be exposed to high pressure and the amplification well may be exposed to a lower pressure so that after source material treatment the source material or a portion of the source material is moves from the high pressure area to the lower pressure area along a pressure gradient that facilitates the source material movement. The transport means may include transport through a filter, chromatography column, hybridization area, or over any adherent material such as plastic, glass, at least one bead, and/or an optical microarray device in order to aid in trapping or purification of the biological target material. A filter may collect cell debris. The transport means may also include chemical reagents, media, probes, or the like.

It is possible that the transport means may include an amplification portion therein so that amplification occurs within the transport means. The source material or a portion of the source material (e.g., the target material) may be pumped from the mixing well through the transport means where it is amplified. Amplification and/or detection reagents necessary to carry out PCR and/or detection may be present during the amplification and thus may be pre-loaded in the amplification well or transferred thereto. The remaining source material or portion of the source material that undergoes amplification may then be pumped into a waste or collection well or tube located within the processing device or external to the processing device. Detection may be integrated into the well (e.g. real-time PCR). Alternatively, other detection methods (such as gel electrophoresis, probe hybridization, or the like) may be performed post-amplification external to or integrated into the processing device. In the event that amplification occurs within the transfer means, the means for providing heat may contact and/or provide heat to the transfer means so that the temperature of the source material or portion of the source material located within the transfer means can be raised and lowered for the amplification process. Further, the transfer means may be include a material that imparts flexibility to the transfer means so that the means can be compressed to minimize the profile width of the transfer means to improve the speed and accuracy of the amplification process.

It is also possible that the mixing well may be composed of or contacted/coated with an adherent material that attracts and/or captures the target material. The adherent may include or be composed of a filter, chromatography column, hybridization area, plastic, glass, at least one bead, immobilized DNA/RNA probe, immobilized antibody, an optical microarray device, or any combination thereof. The mechanical processing (e.g., mixing) may cause sufficient turbulence to contact the target material to the well itself or the adherent material. The adherent material or the target material attached to the well may then be removed from the mixing well and transferred to the amplification portion. The adherent material may be transferred by the transport means to an amplification portion or may be amplified within the transport means. The transfer of the adherent material may be facilitated by a force or means for pulling the material through the transport means. Alternatively, the material may remain within the mixing well with the target material attached thereto and the remaining source material may be pumped out of or removed from the mixing well, thus allowing amplification to take place within the mixing well. A wash step may be incorporated to remove any remaining source material from the target material.

The source material or a portion of the source material may be transferred to a PCR device (e.g., a PCR reaction chamber) such as that disclosed in U.S. Provisional Application No. 61/066,365 filed on Feb. 20, 2008 and PCT Application No. PCT/US09/034446, filed on Feb. 19, 2009 both applications being incorporated by reference herein for all purposes. The amplification process described in the applications referenced above may include positioning some or all of the target material along with one or more PCR reagents between at least two or more opposing spaced apart thermocycling (e.g., thermoelectric) elements that operate by the Peltier effect in a PCR thermal cycling instrument. The PCR device disclosed therein in combination with the simultaneous treatment protocols of the present invention may allow for effective diagnostic testing in less than 2 hours, more preferably less than 0.5 hours and even more preferably less than 0.2 hours.

Some or all of the mixing well content may be transferred to the PCR device manually (e.g., by pipette) or through a transport means such as that described above. Alternatively, the entire processing device or a portion of the processing device (e.g., the mixing well) may be located within the PCR device. The PCR may involve a thermocycling process where the temperature of the target material undergoes a series of temperature increases and decreases in an effort to amplify a desired nucleotide sequence. It is possible that the amplification and detection process may be designed to identify the presence of one or both of sequences IS6110 and IS1081, specific for the M. tuberculosis complex (including M. tuberculosis, M. africanum, M. bovis, and M. microti). The amplification process may detect both sequences so that some strains of M. tuberculosis lacking IS6110 are also detected to provide improved accuracy. Thus, a real-time PCR reaction detecting both IS6110 and IS1081 is advantageous to the process described herein. The teachings herein contemplate the use of one single multiplex reaction wherein the IS6110 and IS1081 molecular beacon probes may be modified with different fluorophores (each exhibiting different emission spectra) to allow for simultaneous detection of both IS6110 and IS1081.

The PCR process may take place in multiple wells so that each well is designed to identify the presence of one or more different sequences, each providing pertinent clinical information into the presence and/or drug resistance of the strain being tested, or any other disease state being identified. For example, the processing device may include a plurality of (e.g., four) amplification wells. For tuberculosis testing specifically, the first well may amplify IS6110 and/or IS1081 to determine the presence of the M. tuberculosis complex. The second well may amplify regions of the rpoB gene and/or provide detection of nucleotide sequences therein that indicate drug sensitivity or resistance to rifampin. Similarly, the third well may amplify regions of the kasG and inhA regulatory region and the ahpC-oxyR intergenic region for isoniazid resistance. However, given the rarity of rifampin mono-resistance, amplification of the rpoB gene that codes for rifampin resistance may be sufficient for diagnosing isoniazid resistance as well. This may assist in minimizing the necessary number of molecular probes. The fourth well may amplify regions of the gyrA gene that codes for fluoroquinolone resistance. Single nucleotide polymorphisms at specific sites in the M. tuberculosis genome have been correlated with rifampin resistance and fluoroquinolone resistance. It is possible that preferably the molecular beacon probes will have the ability to distinguish between targets differing by as little as one nucleotide, and thus the teachings herein envision using such probes for detecting single nucleotide polymorphisms that confer drug resistance. Control reactions (e.g., positive control) may be performed in another well or in an existing well as a multiplex reaction. Wells may be included that amplify other genes which encode resistances to other drugs such as ethambutol or for genes in other organisms such as Clostridium difficle or Staphylococcus aureus.

The amplification and detection processes may involve any process including but not limited to polymerase chain reaction (PCR), reverse transcription polymerase chain reaction (RT-PCR), quantitative real time polymerase chain reaction (Q-PCR), gel electrophoresis, capillary electrophoresis, mass spectrometry, fluorescence detection, ultraviolet spectrometry, DNA hybridization, allele specific polymerase chain reaction, polymerase cycling assembly (PCA), asymmetric polymerase chain reaction, linear after the exponential polymerase chain reaction (LATE-PCR), helicase-dependent amplification (HDA), hot-start polymerase chain reaction, intersequence-specific polymerase chain reaction (ISSR), inverse polymerase chain reaction, ligation mediated polymerase chain reaction, methylation specific polymerase chain reaction (MSP), multiplex polymerase chain reaction, nested polymerase chain reaction, solid phase polymerase chain reaction, or any combination thereof.

The processing device may include a plurality of different wells and/or transfer means such that the type and arrangement of wells and transfer means may be tailored depending on the type of treatment and detection to be performed. The processing device may include one or more mixing wells, PCR wells, detection wells, water wells, reagent wells, waste wells, reverse transcriptase wells, washing wells, or the like. The processing device may also include one or more connecting channels in which a plurality of functions (filtering, hybridization, PCR, detection, or the like) may occur.

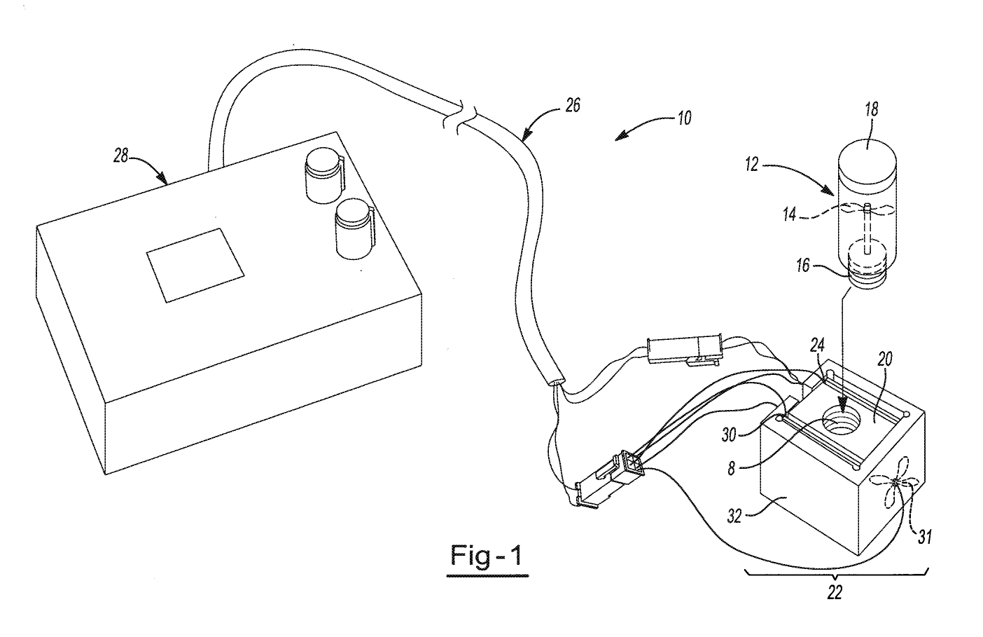

As shown, for example, in FIG. 1, the processing device 10 may include a mixing well 12 having a mixing member 14 therein. The mixing well 12 may be placed between one or more conductive walls 20. The mixing well may further include a motor 16 and a cover 18. The mixing well 12 may include an annular wall and may have a circular top edge as shown. The mixing well 12 may be enclosed within a conductive body 20 that is part of a holding device 22. The conductive body 20 may be adapted to receive the mixing well 12 and may have a complementary shape to the mixing well so that the conductive walls 8 are in direct contact with the mixing well 12. The one or more conductive walls 8 may be contacted by a heating element 24 and/or cooling element 31. The heating element 24 and/or cooling element 31 may be connected to a power source. One or more components of the processing device may be connected to a control device 28 by way of electrical leads 26 which may control the motor speed and direction, the temperature of one or more components of the processing device and the transfer of the source material into, within and out of the processing device. The protocol may be input by the user and the status displayed by way of the control device 28, and the parameters may be changed dynamically in the protocol. The processing device may also include a temperature sensor 30 and an insulating portion 32.

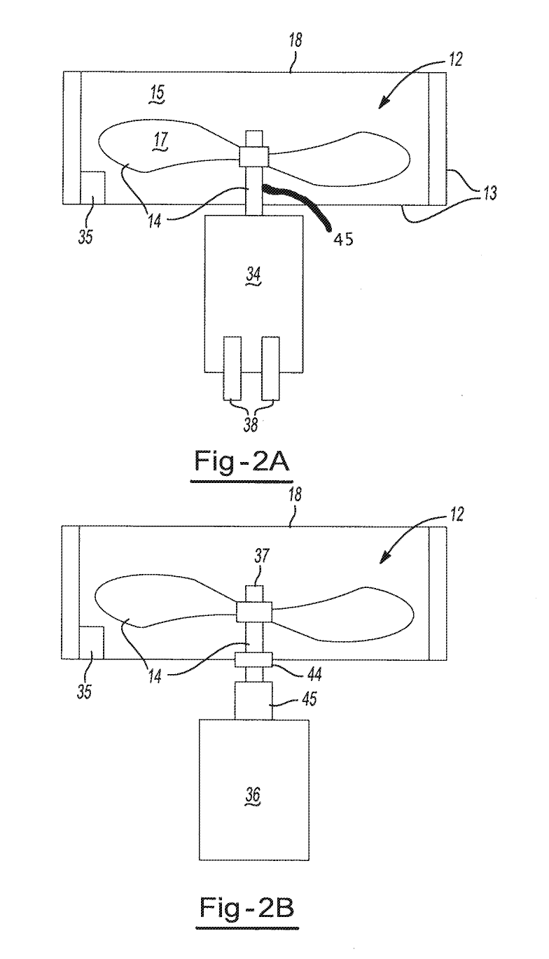

FIGS. 2A and 2B show cross-sectional views of the mixing well 12. FIG. 2A shows a mixing well have an integrated motor 34, whereas FIG. 2B depicts a mixing well having a detachable motor 36. In both FIG. 2A and 2B, the mixing well 12 may include a protrusion or baffle structure 35 extending from a wall of the mixing well 12. FIG. 2A further shows the electrical contacts 38 that connect the internal motor 34 with an external power source. FIG. 2B includes a sleeve portion 44 allowing the motor 36 to attach to the mixing member 14. Both FIGS. 2A and 2B further depict a mixing member 14 attached to the motor 34, 36. In FIG. 2A, the body of the internal motor 34 contacts or is integral to a wall of the mixing well, such that a fluid-tight seal is formed and the motor output shaft 45 may spin freely within the mixing well 12. The motor output shaft 45 is connected to or integrated with the mixing member 14. In FIG. 2B, the external motor output shaft 45 may engage the impeller shaft 37 by way of a cam/key lock engagement. A sleeve/bearing structure 44 is present between a wall of the mixing well and the impeller shaft 37, such that the impeller shaft may spin freely while still providing a fluid-tight seal of the mixing well.

In further reference to FIGS. 2A and 2B, the mixing well 12 is comprised of a body 13, which defines a chamber 15. A cap or cover 18 may be present either as a physical separate piece or integrated with the body 13. Within the chamber 15, a mixing member 14 comprised of blades 17 is mounted to a shaft 37, 45. A sealed electrical motor 34, 36 drives the shaft 37, 45. The top of the sealed motor is joined to the body either by a press-fit or adhesive to further encapsulate the chamber and provide for a fluid tight seal.

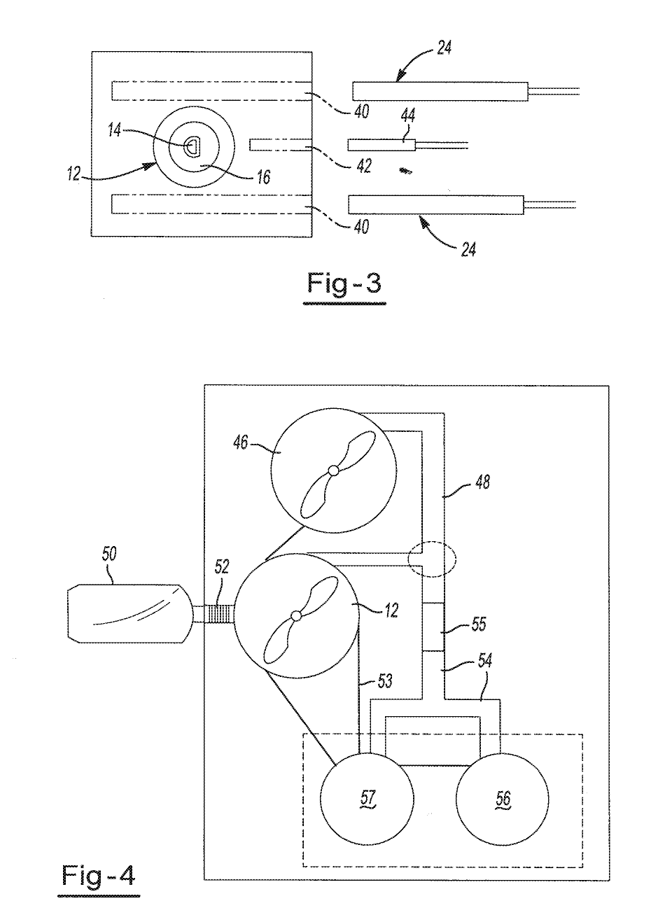

FIG. 3 shows another embodiment of the processing device described herein. A mixing well 12 is shown including a motor 16 and mixing member 14. One or more channels 40 are shown for receiving a heating element 24. The processing device as shown further includes a channel 42 for receiving a temperature sensor 30.

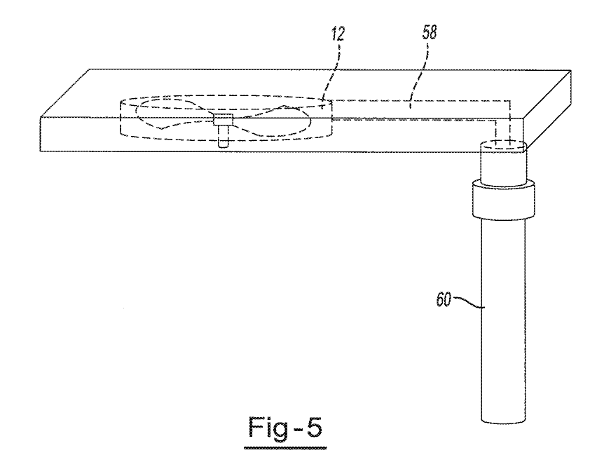

FIG. 4 illustrates a processing device including a reagent well 46 for containing the chemical processing agents prior to chemical treatment of a source material. The processing device further includes a channel 48 for transferring the chemical processing agents to the mixing well 12. Also shown is a specimen container 50 and an interface port 52 for receiving a source material from the specimen container 50. The mixing well may also be attached to a channel 54 for transferring a source material or a portion of the source material to one or more PCR wells 56 or waste wells 57. The channel 54 may further include a transport medium 55 located therein. A channel including a pressure release port 53 may also be included to facilitate movement throughout the device. FIG. 5 illustrates a similar embodiment wherein the mixing well 12 connects to a channel 58 that leads to a tube 60. Amplification and/or detection may occur in the channel 58, tube 60, or both.

Any numerical values recited herein include all values from the lower value to the upper value in increments of one unit provided that there is a separation of at least 2 units between any lower value and any higher value. As an example, if it is stated that the amount of a component or a value of a process variable such as, for example, temperature, pressure, time and the like is, for example, from 1 to 90, preferably from 20 to 80, more preferably from 30 to 70, it is intended that values such as 15 to 85, 22 to 68, 43 to 51, 30 to 32 etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner. As can be seen, the teaching of amounts expressed as “parts by weight” herein also contemplates the same ranges expressed in terms of percent by weight. Thus, an expression in the Detailed Description of the Invention of a range in terms of at “‘x’ parts by weight of the resulting polymeric blend composition” also contemplates a teaching of ranges of same recited amount of “x” in percent by weight of the resulting polymeric blend composition.”

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The disclosures of all articles and references, including patent applications and publications, are incorporated by reference for all purposes. The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of the elements, ingredients, components or steps. By use of the term “may” herein, it is intended that any described attributes that “may” be included are optional.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps. All references herein to elements or metals belonging to a certain Group refer to the Periodic Table of the Elements published and copyrighted by CRC Press, Inc., 1989. Any reference to the Group or Groups shall be to the Group or Groups as reflected in this Periodic Table of the Elements using the IUPAC system for numbering groups.

It will be appreciated that concentrates or dilutions of the amounts recited herein may be employed. In general, the relative proportions of the ingredients recited will remain the same. Thus, by way of example, if the teachings call for 30 parts by weight of a Component A, and 10 parts by weight of a Component B, the skilled artisan will recognize that such teachings also constitute a teaching of the use of Component A and Component B in a relative ratio of 3:1. Teachings of concentrations in the examples may be varied within about 25% (or higher) of the stated values and similar results are expected. Moreover, such compositions of the examples may be employed successfully in the present methods.

It will be appreciated that the above is by way of illustration only. Other ingredients may be employed in any of the compositions disclosed herein, as desired, to achieve the desired resulting characteristics. Examples of other ingredients that may be employed include antibiotics, anesthetics, antihistamines, preservatives, surfactants, antioxidants, unconjugated bile acids, mold inhibitors, nucleic acids, pH adjusters, osmolarity adjusters, or any combination thereof.

It is understood that the above description is intended to be illustrative and not restrictive. Many embodiments as well as many applications besides the examples provided will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventors did not consider such subject matter to be part of the disclosed inventive subject matter.

1. A method for preparing a source material comprising:
introducing a source material into a specimen container;
transferring the source material from the specimen container into a processing device;
treating the source material thermally, mechanically, chemically or any combination thereof within the processing device to alter at least one constitutive characteristic of the source material and to release or create a target material from the source material.

2. The method of claim 1, wherein the step of treating the source material mechanically includes mixing of the source material along with any chemical processing agents in the processing device to promote increased contact between the source material and chemical processing agents for causing cell lysis therein.

3. The method of claim 1, further including a step of amplifying the target material, detecting a target material, or both.

4. The method of claim 1, wherein the step of treating includes the use of a chemical processing agent and a mechanical device that cyclically impinges upon the source material to alter the at least one constitutive characteristic of the source material.

5. The method of claim 1, wherein the step of treating the source material within the processing device includes a step of maintaining the temperature of the processing device at one or more temperatures above room temperature sufficient for mucolysis and cell lysis.

6. The method of claim 1, further including a step of extracting the target material from the treated source material and a step of transferring extracted target material from a first portion of the processing device to a second portion of the processing device that is spaced apart from the first portion.

7. The method of claim 1, further including a step of extracting the target material from the treated source material and a step of transferring extracted target material from a first portion of the processing device to a second portion of the processing device that is spaced apart from the first portion but in fluid communication therewith using a motor-driven member that operates both as a pump and as a mixer.

8. The method of claim 1, further including a step of amplifying the target material by positioning some or all of the target material along with one or more PCR reagents between at least two opposing spaced apart thermoelectric elements that operate by the Peltier effect, in a polymerase chain reaction thermal cycling instrument, thereafter performing a plurality of successive steps of increasing and decreasing temperature of the target material by way of the thermoelectric elements.

9. The method of claim 8, wherein the step of amplifying the target material further includes a step of real-time detection of an attribute of the target material.

10. The method of claim 9, wherein the attribute of the target material is indicative of tuberculosis presence.

11. The method of claim 1, wherein the method is performed at a single point of care medical facility.

12. The method of claim 1, wherein the step of treating the source material chemically includes contacting the source material with one or more chemical processing agents comprising:
a reducing agent selected from the group consisting of dithiothreitol (DTT), mercaptoethanol, mercaptoethylamine, Tris[2-carboxyethyl]phosphine (TCEP), (N-acetylcysteine), Nacystelyn, dornase alfa, thymosin β₄, guaifenesin, or any combination thereof;
a nuclease inhibitor selected from the group consisting of diethyl pyrocarbonate, ethanol, aurintricarboxylic acid (ATA), formamide, vanadyl-ribonucleoside complexes, macaloid, ethylenediamine tetraacetic acid (EDTA), proteinase K, heparin, hydroxylamine-oxygen-cupric ion, bentonite, ammonium sulfate, dithiothreitol (DTT), beta-mercaptoethanol, cysteine, dithioerythritol, tris(2-carboxyethyl)phosphene hydrochloride, a divalent cation such as Mg⁺², Mn⁺², Zn⁺², Fe⁺², Ca⁺², Cu⁺² or any combination thereof; and
a lysis buffer selected from the group consisting of tris-HCl, EDTA, tris-EDTA, EGTA, SDS, deoxycholate, TritonX, NaCl, sodium phosphate, NP-40, phosphate buffered saline (PBS), or any combination thereof.

13. The method of claim 1, further including a step transporting some or all of the target material to an external device for amplification.

14. The method of claim 1, wherein the method takes place in less than 30 minutes.

15. The method of claim 1, wherein the target material is transferred through a capturing medium such as a microporus filter, chromatography column, antibody coated packing or beads, magnetic packing, DNA/RNA probe coated packing, or any combination thereof in order to extract the target material while any remaining source material passes through the capturing medium to assist in purification of the target material.

16. A processing device for biological or chemical samples comprising:
a mixing portion into which a source material is introduced and treated to alter at least one constitutive characteristic of the source material and to release or create a target material from the source material through mechanical, thermal, and chemical treatment;
at least one interface for a control device that controls the temperature, mixing operation, or both within the mixing portion during mixing; and
a covering means for enclosing the mixing portion.

17. The processing device of claim 16, further including an amplification portion for subjecting the target material to amplification by thermocycling.

18. The processing device of claim 17, wherein the amplification portion is optionally integrated with the processing device.

19. The processing device of claim 16, wherein the constitutive characteristic is selected from one or any combination of: rheological (e.g., viscosity), physical (e.g., physically deform or rupture cells), or chemical (e.g. composition, concentration, digestion of suspended solids).

20. The processing device of claim 16, wherein the mixing portion includes a mechanical mixer having a structural member that contacts the source material.

21. The processing device of claim 16, wherein a heater, temperature sensor, and optionally a cooling device are proximate to or included within the mixing portion.

22. The processing device of claim 16, wherein an interface controls or is in communication with one or more heaters, temperature sensors, motors, or any combination thereof.

23. The processing device of claim 16, wherein the mixing portion includes a walled structure defining a cavity that has an opening into which a sample is introduced and an opening into which a motor or impeller shaft passes.

24. The processing device of claim 16, wherein the mixing portion may have one or more ports for the entry and exit of source material, target material, chemical processing agents, or any combination thereof.

25. The processing device of claim 16, wherein the mixing portion has one or more interior wall surfaces adapted to have a sealing fit with a flexible walled bag containing the source material.

26. The processing device of claim 16, wherein multiple processing devices may be used simultaneously under identical mechanical, chemical or thermal treatment conditions, or each may be used simultaneously under different treatment conditions.

27. A processing device for biological or chemical samples comprising:
a device for mixing, pumping, or both;
a body configured to include a processing well adapted to receive the device;
optionally including a fluid transport path optionally including a valve;
at least one heating element and optionally at least one cooling device disposed within the body proximate to or included within the processing well in thermal communication with the device;
a temperature sensing device disposed within the body proximate to or included within the processing well;
a covering for placement over the device so that any contents of the device remain therein.

28. The processing device of claim 27, wherein the processing well or body includes an electric motor with an output shaft that engages an input shaft of the device for mixing and pumping.

29. The processing device of claim 27, wherein the mixing member includes a dual-functional head configured for:
i. homogenization or reducing the viscosity of the contents; and
ii. pumping some or all of the contents from the device to a downstream location.
